(19) United States
(12) Patent Application Publication
(10) Pub. No.: US 2020/0072821 A1
(43) Pub. Date: Mar. 5, 2020

(54) IN VITRO SCREENING AND SELECTION OF SPECIFIC DNA APTAMERS AGAINST THE NON-HUMAN SIALIC ACID, N-GLYCOLYL NEURAMINIC ACID (NEU5GC)

(71) Applicant: National University of Ireland, Galway, Galway (IE)

(72) Inventors: Sharma Rajesh, Galway (IE); Stephen Cunningham, Galway (IE); Joshi Lokesh, Galway (IE)

(21) Appl. No.: 16/489,346

(22) PCT Filed: Feb. 27, 2018
(86) PCT No.: PCT/EP2018/054803

§ 371 Date: Aug. 27, 2019

(30) Foreign Application Priority Data
Feb. 28, 2017 (GB) 1703212.9

(51) Int. Cl. G01N 33/53; C07K 16/28; C12N 15/115; C40B 30/04; G01N 33/532; G01N 33/563

(52) U.S. Cl. CPC G01N 33/5308; C07K 16/28; G01N 33/563; C40B 30/04; G01N 33/532; C12N 15/115

(57) ABSTRACT

The invention relates to methods, assays and compositions for the determination of sialic acid forms. In particular, the invention provides protein and nucleic acid based recognition molecules, for application in the detection, measurement and discrimination of the sialic acid forms N-glycolylneuraminic acid (Neu5Gc) and N-acetylneuraminic acid (Neu5Ac) when presented as either biologically or chemically bound forms or free forms available in suspension.

### FIELD OF THE INVENTION

The present invention relates to methods, assays and compositions for the determination of sialic acid forms. In particular, the invention provides recognition molecules (protein and nucleic acid based), for application in the detection, measurement and discrimination of the sialic acid forms N-glycolylneuraminic acid (Neu5Gc) and N-acetylneuraminic acid (Neu5Ac) when presented as either bound (biological or chemical molecule presented) or free form available in suspension.

### BACKGROUND TO THE INVENTION

Sialic acids are acidic monosaccharides that are found as the terminal monosaccharide on N- and O-linked glycans. They are involved in a range of biological processes, including cell-cell interaction, cell signaling, cell adhesion, and targets for viral infection. Sialic acids have also been implicated in having a role in inflammation, infection and cancer. Predominantly, two forms of sialic acids, N-glycolylneuraminic acid (Neu5Gc) and N-acetylneuraminic acid (Neu5Ac) are found in mammals. Within mammals, Neu5Ac can be converted to its hydroxylated version Neu5Gc by the enzyme cytidine monophosphate N-acetylneuraminic acid hydroxylase (CMAH). However, as a result of an inactivating mutation in the CMAH gene humans lost the ability to synthesize Neu5Gc from Neu5Ac.

The role of Neu5Gc in disease: Sialic acid, specifically Neu5Gc form, has had an ongoing reported association with cancers. First identified as Hanganutziu deicher antigen in patients with serum sickness, Neu5Gc was eventually recognized as an immunogenic antigen conjugated to gangliosides in humans. Though its role and contribution across a range of disorders continues to be investigated, such as its tentative involvement in autoimmune diseases including Hypothyroidism and Hashimoto's thyroiditis.

Neu5Ac acts as a ligand to the receptors promoting cell-cell adhesion in H5N1 and H3N2 viral infections. Also, Plasmodium falciparum receptor recognizes Neu5Ac (Neu5Ac-a(2,3)-Gal) on erythrocytes leading to malaria in humans. In most other mammals (with the exception of humans) Neu5Gc is also widely distributed in addition to Neu5Ac. There are report's suggesting that subtilase cytotoxin (SubAB) produced in E. coli, which is highly toxic to eukaryotic cells causing hemolytic-uremic syndrome has an interaction preference for α2,3 linked Neu5Gc present on the endothelium layer of host cells compared to α2,3 linked Neu5Ac.

Presence of Neu5Gc in Rio-therapeutic preparations: The contamination of recombinant glycoprotein therapeutics expressed in mammalian cell lines such as CHO, NS0 and SP2/0 cells with Neu5Gc (e.g. erythropoietin) has been known for some time. Recent developments and understanding has shown that the contamination of biotherapeutics with non-human glycan epitopes can result in undesirable immunogenic response, as for example occurred with Erbitux (Cetuximab), a recombinant monoclonal antibody preparation produced in murine myeloma cell line. The presence of Neu5Gc on therapeutic protein drugs has further been demonstrated to induce immune complexes in vitro and reducing half-life of drug molecules in mouse models.

Current Methods for Detection and Discrimination of Sialic Acids: Currently analytical method for detection and quantification of sialic acids are based on high-performance liquid chromatography (HPLC) and mass spectrometry (MS). However, these methods require extensive sample preparation (labeling, enzyme digestions, salt removal) for complex samples like serum, highly skilled operators, complex analysis strategies and highly sophisticated instruments. Linkage-specific enzymes (i.e. sialidases, esterases, sia lyases) can all help to define some aspects of the sialic acids on a given glycan of a glycoconjugate. Assay based methods existing for sialic acid analysis employ the use of plant and animal lectins utilized in a number of formats including direct enzyme-linked lectin assay (ELLA). Though lectins from plant and animal sources recognize sialic acids, subject to presentation, their use for in vitro assays are greatly limited due to their poor stability, low sensitivity and cross reactivity with different forms and presentation of sialic acids making it impossible to explicitly discriminate between Neu5Ac and Neu5Gc in such a manner that would be useful for commercial application.

Antibody based detection methods have been developed for Neu5Gc detection in a variety of species including chickens, mice and humans. However all antibody based Neu5Gc detection methods derive the antibodies from crude serum extracts which result in high batch-to-batch variation in terms of both affinity and quality. Serum derived antibodies also appear to have major drawbacks in that their specificity is against the unique Neu5Gc presentation they are raised against. For example, the commercial antibody, Racotumomab, used for treatment of non-small cell lung cancer only recognizes Neu5Gc when attached to the ganglioside GM3 and will fails to recognise Neu5Gc when presented attached to other molecules or free in suspension. Chicken polyclonal antibodies against Neu5Gc have been isolated and used recently to demonstrate an ELISA that had the ability to detect Neu5Gc in recombinant therapeutic glycoproteins. However, there are no reports to date of monoclonal anti-Neu5Gc antibody.

Recombinant anti-sialic acid molecules have been developed by phage display technology, which can recognize both human (Neu5Ac) and non-human (Neu5Gc) sialic acids. Patent application US20110034676 A1 (also published as EP2287202A1) previously described the generation of anti-sialic acid scFv, however, these findings were unable to demonstrate any specificity or discrimination of sialic acid forms (Neu5Gc and Neu5Ac); supporting comparative data provided. Previous to this, patent applications U.S. Ser. No. 10/565,742 and EP20100175798 utilised circulating serum antibodies (whole immunoglobulins) for the indirect assessment of Neu5Ac and a subtractive approach for Neu5Gc concentration within biological solutions. It remains desirable therefore to obtain direct anti-sialic acid molecules for detection, discrimination and measurement.

More recently, aptamers, small DNA/RNA oligonucleotides which can specifically bind target molecules with high specificity have been reported against specific glycan structures including sialyllactose, sialyl Lewis X (sLe^{x}), cellobiose (Glc-β-(1→4)-Glc), and dextran (repeating α-(1→6) glucosidic bonds). Given their stability and chemical flexibility, aptamers have gained significant attention as a source of potentially high affinity and specific recognition elements for use as biosensors and as possible therapeutic agents. A DNA aptamer against Neu5Gc-BSA has been recently described in the literature, utilised in ELISA assay, however, the cross-reactivity of this reported aptamer towards the closely related Neu5Ac has not been explored. A RNA aptamer, has also been reported against Neu5Ac, however, this aptamer has been demonstrated to have high cross-reactivity with Neu5Gc, rendering it unusable in the detection and discrimination of sialic acid forms.

### OBJECT OF THE INVENTION

A first object of the present invention is to provide molecules (i) scFv antibody fragments or (ii) Oligonucleotides DNA aptamers for the detection, monitoring and quantification of sialic acid variants Neu5Gc and Neu5Ac. Although natural anti-Neu5Gc whole immunoglobulin antibodies are present circulating in human serum, these molecules are not suited for in vitro or in vivo applications where the detection of, and measurement of sialic acids is deemed advantageous.

A second object of the invention to permit the detection and quantification of both Neu5Gc and Neu5Ac on biopharmaceutical and recombinant protein preparations produced in recombinant mammalian systems, using ligand binding assay formats, demonstrated here in ELISA format.

A third objective is the use and application of these generated molecules for the affinity capture and the removal of Neu5Gc contaminated molecules (purification) from industrial preparations either during production (in line, at line) of post-production, prior to clinical administration.

A fourth object of this invention is the combined measurement on human derived samples for clinical applications including patient serum profiling and in the field of immunohistochemistry, specifically targeting neural and oncology fields as prognostic and diagnostic tools.

### SUMMARY OF THE INVENTION

According to the present invention there is provided sialic acid recognition molecules which may be (i) a recombinant single chain variable fragment (scFv) antibody or (ii) a DNA oligonucleotide aptamer.

In particular the invention provides sialic acid recognition molecule(s) which are recombinant specific anti-Neu5Gc single chain variable fragment (scFv) antibody fragments comprising a variable light chain sequence and a variable heavy chain sequence. The variable light chain sequence specific for anti-Neu5Gc scFv comprises the following sequences:

Sequence (1): SG-X¹-X₂-X³-X⁴-X⁵-X⁶-X⁷-X⁸-YG, wherein,

X¹ is G or S;

X² is S of H;

X³ is Y or absent;

X⁴ is A or absent;

X⁵ is G or absent;

X⁶ is S or absent;

X⁷ is S or Y or R or G;

X⁸ is Y or A or S,

and Sequence (2): X¹-NDKRPS, wherein,

X¹ is A or S or E or Y,

and either Sequence (3): G-X¹-EDSSGAGHVAI, wherein, X¹ is S or G,

or Sequence (4): GNEDSSDY.

The invention also provides a variable heavy chain sequence specific for anti-Neu5Gc scFv comprising the following sequences:

Sequence (5): GF-X¹-FS-X²-X³-GM-X⁴, wherein,

X¹ is T or D;

X² is D of S;

X³ is Y or H;

X⁴ is M or L or F,

and Sequence (6): YVAAIN-X¹-X²-X³-X⁴-X⁵-T-X⁶-X⁷-G-X⁸-AV, wherein,

X¹ is R or K or S;

X² is H or D or V or I;

X³ is S or G;

X⁴ is E or G or S;

X⁵ is A or Y;

X⁶ is W or Y;

X⁷ is H or Y;

X⁸ is A or P.,

and either Sequence (7): TYYCAKTDY-X¹- GFNVGRID- X²,

wherein, X¹ is S or P; X² is A or P,

or Sequence (8) TYYCAKDS-X¹-SGYH-X²-GRIDA,

wherein, X¹ is S or T; X² is L or I.

Non-discriminant specific sialic acid single chain variable fragment (scFv) antibody fragments, which recognise and bind, capture, Neu5Gc and Neu5Ac sialic acid forms, comprising a variable light chain sequence and a variable heavy chain sequence. The invention also provides total anti-sialic acid single chain variable fragment (scFv) antibody fragments, which recognise, bind and capture, total (both Neu5Gc and Neu5Ac sialic acid forms), comprising a variable light chain sequence and a variable heavy chain sequence.

The variable light chain sequence may comprise at least two of the sequences:
(9) SGGDYGSYYG,
(10) YNDKRPSN, or
(11) GSRDSSYVGI.

The variable heavy chain sequence may comprise at least two of the sequences:
(12) GFTFRNYGMG,
(13) GIYKDGGGTYYAPAL, or
(14) GYATGSSFGDNIDA.

The antibody may further comprise a polypeptide linker sequence, permitting the fusion or linkage of the variable light chain and variable heavy chain sequences. The polypeptide linker may have the formula:
(15) GQSSRSS-Xⁿ(G₄S₂)ⁿ)

wherein, X may be absent or GGGGSS, or GGGGSSGGGGSS.

Thus polypeptide linkers of 7 (GQSSRSS) up to 19 amino acids (GQSSRSS(G₄S₂)ⁿ), where n can be 1 or 2 repeating G4S2 sequences are possible.

In addition the invention provides sialic acid recognition DNA oligonucleotide aptamer molecule(s) which are synthetic nucleic acids specific for either anti-Neu5Gc or anti-Neu5Ac selected from the following sequences which are in a 5′ to 3′ orientation:

anti-Neu5Gc DNA oligonucleotide aptamer molecule(s):
(16) 5′-CGCGCGCAACAATACACACAACCTTCGCGTTCRG-3′,
where R is either G or A,

anti-Neu5Ac DNA oligonucleotide aptamer molecule(s):
(17): 5′-CGCGCCCACATTTAGACCCCAACCTTCGCGGCTTG-3′,

Or (18): 5′-CGCGCCAACAACACCACCAATCTTCGCAGCCCG -3′,

Or (19): 5′-CGCGCGCAACAATACACACAACCTTCGCGTTCGG -3′,

or comprising a consensus sequence:
(20): 5′-SMAMMWHWMSACMMMWMYYYBSVSBBCK -3′,
where S is either C or G; M is either C or A; W is either T or A; H is either T, C or A;
Y is either C or T; B is either T, G or C; V is either G, A or C; K is either T or G.

In a further aspect the invention provides nucleic acid or oligonucleotide sequences selected from those identified above and sequences which are substantially similar to the said sequences and which also bind to Neu5Gc and/or Neu5Ac. In particular the invention provides nucleic acid sequences having at least 85% sequence identity with these sequences, more particularly sequences having at least 90% or at least 95%, or at least 97%, or at least 98%, or at least 99% sequence identity with the sequences. The sequences may also be substantially complimentary to the above sequences, and having the same percentage complementarity as set out above for sequence identity. All sequence identities are those measured under high stringency conditions.

The invention also provides a method of detection, monitoring and/or quantification of the sialic acid variants Neu5Gc and Neu5Ac, comprising use of a recombinant single chain variable fragment (scFv) antibody as defined above, or an oligonucleotide aptamer sequence as defined above. In particular such methods, utilising a ligand binding assay such as immunoassay (e.g. ELISA), may be used to detect and quantify both variants in clinical and animal samples, biopharmaceutical processes, biotherapeutic preparations and recombinant protein batches.

Also provided is a method of determination of total sialic acid comprising use of a recombinant single chain variable fragment (scFv) antibody as defined above, or an oligonucleotide aptamer sequence as defined above, wherein, the determination of total sialic acid (Neu5Ac and Neu5Gc) and the determination of Neu5Gc content specifically, will permit the determination of (i) Neu5Gc, (ii) Total sialic acid and (iii) Neu5Ac. In particular such methods, utilising ligand binding assays such as an immunoassay, measurement of total sialic acid and Neu5Gc would be performed in parallel with inferred quantification Neu5Ac determined by subtraction.

The invention provides a method for the removal of Neu5Gc contaminated molecules from industrial or pharmaceutical preparations, achieved by the affinity capture of contaminated molecules from the preparations either during production or post-production, upstream of clinical or final use. By presenting the scFv on an affinity matrix, resin and passing contaminated sample for purification across.

Also provided is a bio assay kit comprising an oligonucleotide as defined above, or an antibody as defined above.

The scFv molecules and aptamer molecules may be further directly labelled or conjugated with a detectable label. Modifications of scFv and or aptamer molecules are within the scope of this invention and may be “chemically modified or labelled” and this could include, but is not limited to, chemiluminescent, bio luminescent, fluorescent, electroactive, mass added or magnetic labels all of which are not found in nature.

The advantage of the present invention is that the methods, assays and compositions are capable of recognising_(Neu5Gc) in both free/released and protein bound form, which permits detecting detection and quantification of the molecule. In addition the invention permits detection of total sialic acid content i.e. combined (Neu5Gc) and (Neu5Ac) in both protein bound and free/released forms. As a result a combination of both determinations permits the inferred quantification of (Neu5Ac) content.

### BRIEF DESCRIPTION OF THE DRAWINGS

FIG. 1: Serum response to immune challenge with Neu5Gc conjugates determined by direct ELISA, with serum dilution range from 1/10,000 to 1/320,000.

FIG. 2: Demonstration of the full length scFv fragment amplicons (Top) and demonstration of diversity of library across randomly selected clones examined by restriction digestion using BstOI (Bottom). Shown on 1.5% agarose gel.

FIG. 3: Amplified phage pools from short and long-linker scFv-phage display library before (pre-pan) and after three rounds of selective panning The panning products were analysed for enrichment of anti-Neu5Gc polyclonal scFv displaying phage pool on an antigen coated (Neu5Gc-PAA, 2.5 μg/mL) plate via indirect ELISA, detected with anti-M13-HRP secondary antibody. Signal obtained was compared against assay controls (Neu5Ac-PAA and HSA). Indirect ELISA revealed enrichment of anti-Neu5Gc scFv-phage population from round 2 onwards.

FIG. 4: Analysis of 49 isolated individual scFv clones from pan 3 of the short-linker (SL) library. Each scFv-phage clone was applied to antigen coated (Neu5Gc-PAA, 2.5 μg/mL) 96 well microtiter plate blocked with 0.5% HSA in PBS and incubated for 1 hour at 37° C. The plate was washed with PBS-T 3 times and bound scFv-phage detected with anti-M13-HRP conjugated antibody. The signal obtained was compared against assay control Neu5Ac-PAA.

FIG. 5: Analysis of 21 isolated individual scFv clones from pan 3 of the long-linker (LL) library. Each scFv clone was applied to antigen coated (Neu5Gc-PAA, 2.5 μg/mL) 96 well microtiter plate blocked with 0.5% HSA in PBS and incubated for 1 hour at 37° C. Plate was washed with PBS-T 3 times and bound phage detected with anti-M13-HRP conjugated antibody. The signal obtained was compared against assay control Neu5Ac-PAA.

FIG. 6: Reducing 12% SDS-PAGE with silver staining showing increasing purity of NUIG_SL1A1 during purification process. Lane 1: Molecular mass marker, lane 2: crude lysate, lane 3: flow-through, lane 4: Buffer A wash, lane 5: 10% B wash, lane 6 to lane 8: Elution fractions.

FIG. 7: Purified soluble scFv (NUIG_SL1A1, NUIG_SL4D1 and NUIG_LL1B6, analysed by direct ELISA against Neu5Gc.

FIG. 8: Comparison of inhibition curves for NUIG_SL1A1, NUIG_SL4D1 and NUIG_LL1B6 scFv's performed by ELISA. Error bars indicate ±SD.

FIG. 9: Specificity of NUIG_SL1A1 to Neu5Gc sialic acid. (A) Free Neu5Gc showing concentration dependent inhibition of scFv. (B) Free Neu5Ac and (C) Gal-α-(1,3)-Gal failed to inhibit scFv binding to immobilised Neu5Gc. (D) Neu5Gc displayed on bovine fetuin showing concentration dependent inhibition of scFv. (E) Weak inhibition response observed with asialofetuin. The scFv was inhibited by free Neu5Gc and glycoprotein bound Neu5Gc (bovine fetuin) while no inhibition was observed with rest of the negative controls indicative of Neu5Gc specific binding of scFv.

FIG. 10: Representative ‘Composite Curve’ for anti-Neu5Gc NUIG_SL1A1. Composite curve from combined independent replicate assays demonstrating reproducibility of inhibition assay. Error bars indicate ±SEM.

FIG. 11: Immuno Blotting using PVDF membrane. NUIG_SL1A1 used to detect Neu5Gc over bovine transferrin and bovine fetuin treated with acid to remove sialic acids. Lane 1 and 6: Molecular mass marker, lane 2: 1 μg of non-acid treated bovine fetuin, lane 3-5: 1, 2, 4 μg of acid treated bovine fetuin, lane 7: 1 μg of non-acid treated bovine transferrin, lane 8-10: 1, 2, 4 μg of acid treated bovine transferrin. Neu5Gc detected over non-acid treated glycoproteins, no signal was obtained for Neu5Gc after removal of sialic acid by acid treatment.

FIG. 12: SELEX enrichment of ssDNA pools against Neu5Gc-sp-biotin obtained over 10 rounds of biopanning

FIG. 13: SELEX enrichment of ssDNA pools against Neu5Ac-sp-biotin obtained over 10 rounds of biopanning

FIG. 14: Specific binding profile against Neu5Gc of aptamers NUIG GC-224, NUIG GC-239 and a negative control antisense aptamer against biotinylated Neu5Gc-sp-biotin [2 uM] and sp-biotin [2 uM]. 500 nM of aptamer was used throughout.

FIG. 15: Eluted ssDNA aptamer NUIG GC-224 from a bead based binding assay, ran out on urea PAGE. Lane 1=Neu5Ac-sp-biotin; Lane 2=Neu5Gc-sp-biotin; Lane 3=sp-biotin. 25 bp DNA ladder is ran along side, ssDNA bands of 60 bp size are visible between 50 bp and 75 bp ladder. Signal recovery in lane 2 can be observed.

FIG. 16: Concentration cover to saturation for detection curve generation of NUIG_GC-224 against Neu5Gc. Concentration range from 0 to 625 nM of aptamer tested against 250 pmol (constant) of Neu5Gc-spacer-biotin presented immobilized on dynabeads.

FIG. 17: Neu5Gc detection using NUIG_GC-224. Four samples, two protein (fetuin and asialofetuin) and two serum (human and mouse) were biotinylated prior to immobilisation on to streptavidin dynabeads. Aptamer concentration used for the binding and detection of Neu5Gc, 500 nM constant.

FIG. 18: Inhibition profile, ‘Inhibition Curve’ of NUIG_GC-224 (500 nM, constant) against Neu5Gc-sp-biotin (250 pmol, constant) using free Neu5Gc and free Neu5Ac monosaccharides ranging from 0-1 mg/mL across eight points (represented as Log (10) ng/mL on x-axis).

FIG. 19: Purified scFv fragments NUIG_SL1A1, NUIG_SL4D1, NUIG_LL1B6, NUIG_LL4E2, NUIG_LLY and NUIG_LL_AC/GC (also denoted as NUIG_LLZ) tested in direct ELISA against Neu5Gc and Neu5Ac presented as PAA conjugates. ELISA were kept constant, Blocking was performed using periodate treated BSA, a 1/10000 dilution of 1 mg/ml scFv preparation was used for each scFv, Tween-20 was contained within all PBS wash buffers. Detection was performed using an anti-HIS HRP secondary and TMB substrate.

FIG. 20: Purified scFv fragments NUIG_SL1A1, NUIG_SL4D1, NUIG_LL1B6, NUIG_LL4E2, NUIG_LLY and NUIG_LL_AC/GC (also denoted as NUIG_LLZ) tested in direct ELISA against sialoglcoprotein Transferrin from Human and Bovine sources. Human transferrin would only be expected to be observed with Neu5Ac sialic acid, while Bovine transferring would carry both Neu5Ac and also Neu5Gc. ELISA were kept constant, using 5 ug/ml of each respective transferrin, blocking was performed using periodate treated BSA, a 1/10000 dilution of 1 mg/ml scFv preparation was used for each scFv, Tween-20 was contained within all PBS wash buffers. Detection was performed using an anti-HIS HRP secondary and TMB substrate.

FIG. 21: Composite curves generated from Direct ELISA using free Neu5Gc against scFv binding to a fixed antigen (5 ug/mL Bovine Transferrin) Inhibition range used throughout was from zero up to 100,000 μM free Neu5Gc Inhibition was observed for the 5 anti-Neu5Gc scFv as expected, with IC50 values ranging from 300 to 10000 μM.

### TABLE LEGENDS

Table 1: Alignment of CDR regions of anti-Neu5Gc specific scFv
Table 2: Alignment of CDR regions of anti-sialic acid (Neu5Ac and Neu5Gc) scFv
Table 3: Conditions of SELEX rounds 1 to 10 for selection of Neu5Gc specific binding DNA aptamers.
Table 4: Conditions of SELEX rounds 1 to 10 for selection of Neu5Ac specific binding DNA aptamers
Table 5: Alignment of 3 DNA aptamers, NUIG_Gc-101, NUIG_Gc-111 and NUIG_Gc-224, and their percentages returned post SELEX sequencing demonstrating specificity to Neu5Gc. Consensus sequence is derived.
Table 6: Alignment of 3 DNA aptamers, NUIG_Ac-79, NUIG_Ac-89-94 and NUIG_Ac-93-68, and their percentages returned post SELEX sequencing demonstrating specificity to Neu5Gc. Consensus sequence is derived.

### DETAILED DESCRIPTION OF THE DRAWINGS

High Specificity scFv Generation, Isolation and Application

Immunisation of Chickens: Adult male Leghorn chickens were immunized by intramuscular injection with 50 μg of the glycoconjugate preparation (Neu5Gc containing neoglycoconjugates (Neu5Gc-BSA) and glycoconjugates (Neu5Gc-GM3)), in a total volume of 400 μL, over a series of 3 immunisations.

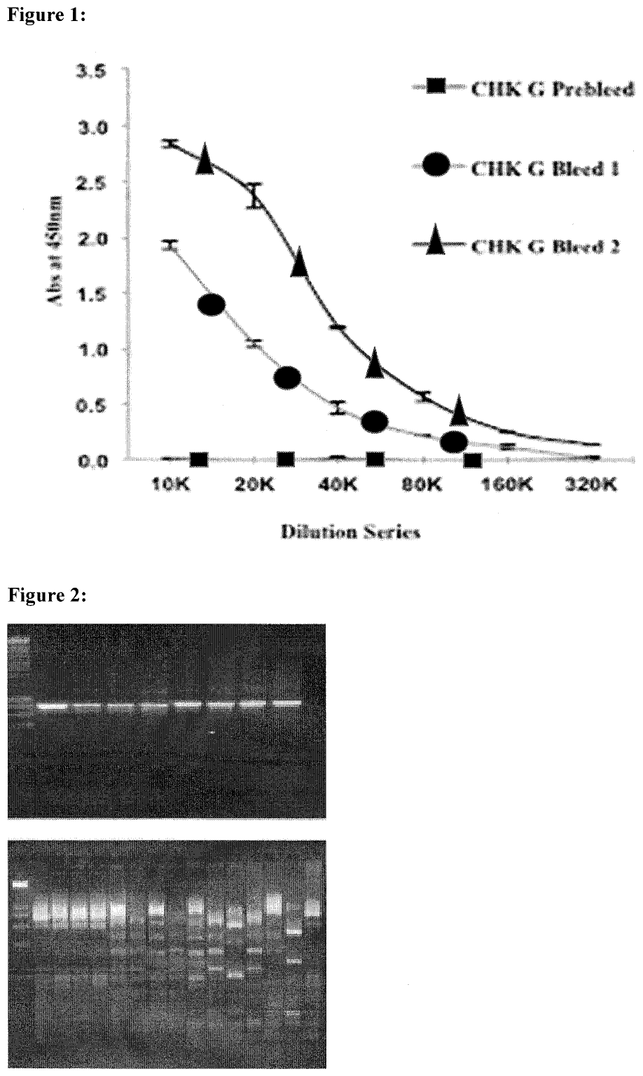

Detection of immune response to Neu5Gc sialic acid: Serum Neu5Gc response was evaluated by direct enzyme-linked immunosorbent assay (ELISA) analysis, FIG. 1. Briefly, 96-well immunoassay plates (Maxisorp; Nunc) were coated overnight at 4° C. with 100 μL/well Neu5Gc-PAA (Lectinity, Russia) at 2.5 μg/mL. Wells were blocked with 0.5% periodated BSA (pBSA) in phosphate buffer saline pH7.4 (PBS-B) for at 37° C. four 1 hour, washed thoroughly with phosphate buffer saline containing 0.05% Tween 20 (PBS-T). Sera samples were serially diluted from 10,000× to 320,000× in 0.1% pBSA in PBS-T (PBS-BT) and added to the plates (100 μl/well). Plates were then incubated at 37° C. for 1 h, and then washed three times with PBS-T, and probed for 1 hour at 37° C. with horseradish peroxidase (HRP)-conjugated anti-chicken IgG antibody (ThermoFisher, UK) in PBS-BT. After washing with PBS-T, the wells were developed with 100 μL/well HRP substrate, tetramethylbenzidine (TMB), (ThermoFisher, UK), and reactions were stopped with 100 μL/well 1 M H₂SO₄. All analyses were performed in triplicate.

Competitive Inhibition ELISA with free Neu5Gc: To ensure that the polyclonal response was directed towards the Neu5Gc, and no other component of the glycoconjugate preparation utilised for immunization, the polyclonal serum was also examined by competitive inhibition, using free Neu5Gc in a competitive ELISA format. Briefly, a 96-well immunoassay plates (Maxisorp; Nunc, UK.) were coated overnight at 4° C. with 100 μL/well Neu5Gc-PAA (Lectinity, Russia) at 2.5 μg/mL, followed by blocking with 0.5% pBSA in PBS pH 7.4, and then washed 3 times with PBS-T. Diluted polyclonal serum (1:40,000) was used to probe Neu5Gc in presence of free Neu5Gc at a range of concentrations, creating competition for binding between immobilised and free Neu5Gc from 1 μg/mL (3.75 μM) to 10 mg/mL (30.7 mM) in 0.1% pBSA in PBS pH7.4. Throughout, controls used were Neu5Ac and Gal-α-(1,3)-Gal both sourced from Dextra, UK and proteins fetuin and asialofetuin, both bovine, sourced from Sigma-Aldrich. All were tested at 1 μg/mL to 10 mg/mL in pBSA in PBS pH 7.4. Plates were then incubated at 37° C. for 1 hour, and then washed three times with PBS-T, and probed for 1 hour at 37° C. with horseradish peroxidase (HRP)-conjugated anti-chicken IgG antibody (ThermoFisher, UK) in PBS-BT. After washing with PBS-T, the wells were developed with 100 μL/well HRP substrate, TMB (ThermoFisher, UK), and reactions were stopped with 100 μL/well 1 M H₂SO₄. All analyses were performed in triplicate. A reduction in assay signal corresponds to a reduction in binding, and was estimated as percentage of binding compared to a non-competitor control. A plot of percentage binding at free Neu5Gc concentration generated an inhibition curve.

Chicken scFv Library Generation: Library generation and phage display was performed using the protocols as described by Andris-Widhopf et al., 2000. Briefly, total RNA was isolated from the spleen and bone marrow of one femur from each chicken (TRIzol Reagent; Invitrogen Inc., USA) and first-strand cDNA synthesised (Superscript III; Invitrogen Inc., USA) both as per manufacturers protocol. The scFv libraries were generated using a two-step approach with initial amplification of heavy and light chains, followed by overlap extension PCR. Libraries corresponding to both short and long linker regions were produced in this manner. The scFv cDNA products introduced into the pComb3 phagmid system via ligation and subsequently transformed into electrocompetent E. coli XL-1 Blue cells (recAl endAl gyrA96 thi-1 hsdR17 supE44 relAl lac [F′, proABlacIqZΔM15 Tn10 (Tetr)]) (Agilent Technologies, USA). Total library size were estimated by antibiotic resistance plate counting on Luria-Bertani (LB) agar containing 100 μg/ml ampicillin (Sigma-Aldrich, Germany). scFv insertion was validated by colony PCR and library sequence diversity assessed using endonuclease BstNI (New England Biolabs, USA) digestion of PCR products and visualised by agarose gel electrophoresis, FIG. 2. The completed library preparations were propagated in XL-1 Blue cells, and held as glycerol stocks.

Production of scFv-displaying phage. To produce and isolate scFv-displaying phage, 100 mL super broth (SB) supplemented with 2% glucose, 100 μg/mL ampicillin and 10 μg/mL tetracycline was inoculated with approximately 2×10⁹ cells from the glycerol stock library. The culture was then incubated with rotation at 37° C. until an OD₆oo of 0.5, was reached. At this point the culture is co-infected with 10¹⁰ pfu/mL colony forming unit of VCSM13 helper phage (Agilent Technologies, USA) and increased to a volume of 200 mL with SB. After 90 minutes incubation, 50 μg/mL kanamycin was added and incubation 37° C. overnight carried out. Phage particles were purified and concentrated from the liquid medium by PEG/NaCl precipitation and resuspended in 0.5% PBS-B and passed through a 0.22 μM syringe filter. Recovered phage particles where presented for biopanning

Selection by biopanning of scFv-phage pools against Neu5Gc. Biopanning against Neu5GC was performed, for both linker length libraries, using Maxisorp immunotubes (Nunc, UK.). Briefly, tubes were coated overnight at 4° C. with 1 mL of 10 μg/mL Neu5Gc-PAA (Lectinity, Russia) and then blocked with 0.5% PBS-B for 1 hour. For negative selection, immunotubes were only blocked with 0.5% PBS-B without receiving any Neu5Gc conjugate coating. For negative selection 1×10¹¹-10¹² scFv-displaying phage suspended in PBS-B were then added and mixed by rotation for 1 hour at room temperature. Follow by repeated washing with PBS-T, and then with PBS only. The unbound phage, were removed and directly exposed to antigen coated Neu5Gc-PAA, immunotubes tube for 1 hour at 37° C. Post incubation, immunotubes were washed Tris Buffer Saline pH 7.5 (TBS) containing 0.05% (v/v) Tween 20, TBS-T (Sigma-Aldrich, Germany). Bound phage were eluted with the addition of 1 mL 100 mM glycine-HCl, pH 2.2 for 10 min. Eluted phage were neutralized using 500 μL of 1M Tris-HCl, pH 8.8. Propagation of eluted phage was then performed. Three rounds of panning were performed, with increasing selection stringency mediated by a progressive increase in washing steps from 10 in pan 1 to 15 in pan 2 and 3.

Direct ELISA analysis of the scFv-phage population polyclonal phage: After each round of biopanning was performed, Maxisorp immunoplates (Nunc, UK) were coated with either 2.5 ug/mL of Neu5Gc-PAA (Lectinity, Russia) as antigen positive or Neu5Ac-PAA (Lectinity, Russia) as selection negative control, and blocked with 0.5% human serum albumin (HSA) in PBS, followed by washing with PBS-T. Threefold dilution of phage-scFv preparations (in 0.1% HSA in PBS) were added to the wells in 100 uL volumes. After 1 hour of incubation at 37° C., plates were washed with three times with PBS-T and probed with HRP-conjugated anti-M13 antibody (GE Healthcare, Germany) in PBS-BT for 1 hour at 37° C. After extensive washing with distilled water, the wells were developed as described above.

Isolation and analysis of single scFv-phage clones: From the third round of panning, individual clones where cultured in 96-well culture block (Sarstedt, Germany) in 1 mL of SB media supplemented 1% (v/v) glucose, ampicillin and tetracycline at 100 ug/mL and 10 ug/mL respectively. The cultures were grown overnight shaking at 200 rpm at 37° C., following overnight growth the culture was then sub-cultured, inoculum of 10 uL into 1 mL of fresh SB media supplemented with 1% (v/v) glucose, ampicillin and tetracycline as before. The culture was grown up to log phase, VCSM13 helper phage (˜10¹⁰ particles) was then added for phage particle formation and culture was grown for 2 hours shaking at 200 rpm at 37° C. After incubation, kanamycin was added to 50 ug/mL and culture was grown overnight shaking at 200 rpm at 37° C. Subsequent day the culture was centrifuged at 3,300 g for 30 minutes at 4° C. The pellet was presented for plasmid (phagemid) extraction (NucleoBond, Macherey-Nagel) and the supernatant was presented for PEG/NaCl phage particle precipitation. Isolated individual phage particle were analysed for interaction and binding to Neu5Gc by direct ELISA. Briefly, Maxisorp immunoplates were coated with 2.5 ug/mL of Neu5Gc-PAA as antigen and Neu5Ac-PAA as assay control in 100 mM bicarbonate buffer pH 9.6 overnight at 4° C. The immunoplate was then washed with PBS, pH 7.4 and blocked with 0.5% human serum albumin (HSA) (Sigma-Aldrich, Germany) in PBS, pH 7.4 for 1 hour at 37° C., after which the plate was washed with PBS-T. The PEG/NaCl isolated individual phage-particles were then tested in triplicate. The plate was incubated for 1 hour at 37° C., following which the plates were washed with PBS-T. HRP-conjugated anti-M13 antibody at 1:5,000 dilution in PBST was added (100 μL/well) and incubated for 1 hour at 37° C. After incubation the plate was washed with PBS-T, followed by addition of HRP substrate, TMB (ThermoFisher, UK) 100 uL/well and incubated in dark for 20 min at room temperature. The reaction was stopped with 100 μL/well 1 M H₂SO₄.

Sequence analysis of single scFv clones: Individual clones selected from third round of panning were sequencing (Eurofins MWG Operon). Multiple sequence alignment, translation, and recognition of complementarity-determining regions (CDRs) was performed using BLAST (http://www.ncbi.nlm.nih.gov/BLAST), ClustalW2 from EBI (http://www.ebi.ac.uk/Tools/msa/), and the CDR repository held at http://www.bioinforg.uk/abs.

Expression and Purification of soluble scFv: Based on the observed interaction of individual scFv-phage particles and the clustering permitted with sequence information, representative single clones for each unique sequence was presented for soluble scFv expression. Phagemid preparations were transformed by heat shock into E. coli strain TOP 10F′ (Invitrogen, USA) and were then plated on Luria-Bertani (LB) agar with ampicillin 100 ug/mL and tetracycline 10 ug/mL, to permit growth of single colonies. Picked colonies were cultured overnight in 20 mL SB media containing 100 ug/mL ampicillin and 10 μg/mL of tetracycline (37° C., 250 rpm shaking). Overnight culture was diluted 1:50 in 200 mL of LB media with 100 μg/mL ampicillin and 10 μg/mL of tetracycline, culture was grown shaking at 200 rpm at 37° C. until log phase, OD₆₀₀ of ˜0.6 was achieved. Soluble expression was induced upon addition of 1 mM isopropyl-β-D-thiogalactoside, post induction the culture was grown overnight shaking at 200 rpm at 37° C. To isolate soluble scFv, bacterial cells were pelleted by centrifugation at 3,500×g for 20 minutes at 4° C. and resuspended in equilibration buffer (50 mM Na₂HPO₄, 250 mM NaCl, 10 mM imidazole, pH 7.4). Periplasmic scFv was then released by sonication, cell debris was removed by centrifugation at 10,000×g for 30 minutes at 4° C., pellet was discarded and the supernatant was filtered through a 0.45 μm (Sarstedt, Germany) followed by 0.22 μm filtration (Sarstedt, Germany). Soluble scFv antibodies were purified by nickel chelation chromatography (Ni-NTA Superflow, Qiagen). For large scale production, culture volumes were increased to litre scale, with purification performed using an AKTA purifier FPLC under naïve conditions using HisTrap HP columns (GE Healthcare, Germany) using ten column washes and a linear gradient up to 250 mM imidazole for all elutions. Purified samples were dialysed against PBS and concentrated by membrane filtration (5 kDa filtration columns; Vivascience, Mass.). Protein concentration on final products was estimated by BCA assay (Thermo fisher, UK). Purified scFv were stored at a concentration of 1 mg/mL at 4° C.

Evaluation of purified soluble scFv by ELISA: Neu5Gc binding of soluble scFv was evaluated by direct ELISA. 2.5 ug/mL of Neu5Gc-PAA (Lectinity, Russia) as antigen and Neu5Ac-PAA (Lectinity, Russia) as assay control in 100 mM bicarbonate buffer pH 9.6 were used to coat a maxisorp immunoplate (100 uL/well) overnight at 4° C. The plate was washed with PBS. Wells were blocked with PBS-B for 1 hour at 37° C., followed by washing with PBS-T. Clarified cell lysate was then applied to each well (100 uL/well) and incubated for 1 hour at 37° C. Post incubation, plate was washed with PBS-T. Anti-6× His-HRP secondary at 1:10,000 dilution in PBS-T was added (100 μL/well) and incubated for 1 hour at 37° C. After incubation the plate was washed with PBS-T, followed by addition of HRP substrate, TMB and incubated in dark for 20 minutes at room temperature. The reaction was stopped with 100 μL/well 1 M H₂SO₄.

Development of scFv-based competitive ELISAs: For competitive ELISA 96 well immunoassay plates (Maxisorb, Nunc) were coated (100 uL/well) overnight at 4° C. with 2.5 ug/mL of Neu5Gc-PAA (Lectinity, Russia) in 100 mM bicarbonate buffer pH9.6. The plate was then washed with PBS. Wells were blocked with 0.5% HSA in PBS for 1 hour at 37 ° C. The plate was washed PBS-T. For competition, Neu5Gc free sugar was diluted from 10 mg/mL (30.7 mM) to 1 μg/mL (3.75 μM) in 0.1% HSA in PBS.

As competitive assay 50 uL of the free sugar solution, of known concentration was added, along with 50 uL of a 1:40,000 diluted anti-sialic acid scFv (produced as soluble scfv protein) to microtiter plate wells in triplicate. As assay (non-competitive) controls Neu5Ac and Gal-α-(1,3)-Gal (Dextra, UK) and glycoproteins fetuin and asialofetuin (Sigma-Aldrich, USA) in 0.1% HSA PBS were used. After addition of standard and controls, plate was incubated at 37° C. for 1 hour. Post incubation, the plate was washed with PBS-T. Anti-6× His-HRP secondary at 1:10000 dilution in PBS-T was added (100W/well) and incubated for 1 hour at 37° C. After which the plate was washed (PBS-T) and addition of HRP substrate (TMB) carried out at 100 uL/well. Reaction was permitted to run in the dark for 20 minutes at room temperature, stopped upon the addition of 1 M H₂SO₄.

Surface Plasmon Resonance: The sialic acid binding and discrimination ability of selected clones were assessed using surface plasmon resonance (SPR) utilizing a Reichert 7000 double channel instrument. All steps were performed in PBST (10 mM PO₄, 2.4 mM KCl, 138 mM NaCl, 0.05% Tween-20 at pH 7.4). Chemistry for functionalization was 0.4 M 1-ethyl-3-(3-dimethylamino-propyl):carbodiimide hydrochloride: 0.1 M N-hydroxysuccinimide as standard throughout, with chip capping 1.0 M ethanolamine-HCl, pH 8.5. Association and dissociation rate constants were fitted using algorithm constants within Scrubber analysis software.

Detection of Neu5Gc on membrane transfer biological sample: scFv SL1A1 was tested for detection of Neu5Gc on membrane transferred, western blotted, protein preparations. A panel of proteins and glycoproteins comprising HSA, BSA, pBSA, OSA, bovine transferrin, human transferrin, human alpha-1 -acid glycoprotein, asialofetuin and fetuin were all western blotted onto a PVDF membrane post SDS-PAGE. The PVDF membrane was blocked with 2% fish gelatin (Sigma-Aldrich, USA) in PBS for 2 hours at room temperature, followed by washing with PBS-T. Purified soluble scFv at 10 μg/mL in PBST was used as a primary detection molecule for Neu5Gc, incubated for 1 hour at room temperature. Post incubation, the membrane was washed with PBS-T, and an anti-HIS-HRP conjugated secondary antibody at 1:10,000 dilution in PBST then used to permit the detection of bound scFv when developed with BCIP/NBT substrate (Sigma-Aldrich, USA).

II. DNA Aptamer Selection, Identification and Application

Random DNA library and amplification oligonucleotides: A random DNA library, comprising of two conserved flanking regions of 15 nucleotides, harbouring a restriction site for digestion, with a 30-N nucleotide random sequence positioned between. The set of oligonucleotides used for the library amplification were; sense: 5′-[Biotin]GCGCGGATCCCGCGC-3′ and antisense: 5′-GCGCAAGCTTCGCGC-3′. 10 nM of the synthesized DNA library was amplified by polymerase chain reaction (PCR) under the following conditions: 20-25 cycles of 20 seconds at 94° C., 40 seconds at 55° C. and 40 seconds at 75° C. A biotinylated sense oligonucleotide was used throughout PCR reactions for the generation of biotinylated double stranded DNA (dsDNA). Biotinylated dsDNA was then incubated with streptavidin (1:10 molar ratio) for 1 hour at room temperature, followed by denaturation, presenting single stranded DNA (ssDNA). Streptavidin bound biotinylated ssDNA was then separated from non-biotinylated strands by Urea-PAGE (NuPage, Invitrogen, USA) and extracted using crush and soak method followed by ethanol precipitation.

In vitro selection of Neu5Gc and Neu5Ac specific aptamers: Indirect capture method was used for screening of aptamers against (i) Neu5Gc-sp-biotin and (ii) Neu5Ac-sp-biotin. Prior to selection, a counter selection (background removal) against sp-biotin was performed. Briefly, ssDNA pool (˜400 ng) in binding buffer (10 mM phosphate buffer, 2.7 mM potassium chloride and 137 mM sodium chloride, pH 7.4), was heated at 95° C. for 5 minutes, then snap cooled on ice for 10 minutes and subsequently incubated at room temperature for 10 minutes. To this suspension, 100 pmol of sp-biotin was added and the volume made up to 500 uL with binding buffer, followed by incubation at 37° C. for 1 hour to permit ssDNA:sp-biotin complex formation and interaction, after which the sp-biotin was immobilized by capture on 250 ₗug of MyOne streptavidin C1 Dynabeads. The beads coated with sp-biotin and interacting ssDNA complexes were separated from suspension by magnetic pulldown, and unbound, non-interacting ssDNA were desalted and concentrated by phenol-chloroform extraction for amplification and presentation for positive selection against (i) Neu5Gc-sp-biotin or (ii) Neu5Ac-sp-biotin. The selection protocol for target glycan conjugates was as described above with the exception of either (i) Neu5Gc-sp-biotin or (ii) Neu5Ac-sp-biotin being presented, bound ssDNA was eluted directly from the recovered MyOne Streptavidin C1 Dynabeads (Invitrogen), twice with using nuclease free water and heating at 95° C. for 5 minutes to disrupt bound complex, interaction. The eluted ssDNA was desalted, PCR amplified, converted into ssDNA pool, and used for subsequent SELEX rounds. The stringency during aptamer selection was heightened gradually by decreasing Neu5Gc-sp-biotin concentration (100 to 50 pmol) and interaction exposure time (60 to 25 minutes), and by increasing salt concentrations of binding buffer. Two negative/counter selections were performed during the selection process. Details of selection regime and rounds of panning against (i) Neu5Gc-sp-biotin and (ii) Neu5Ac-sp-biotin are provided in Table 3 and Table 4 respectively.

Phenol-Chloroform Extraction of ssDNA: ssDNA bands were excised from urea gel and diffused at 50° C. for 30 minutes in double volume of diffusion buffer (0.5 M Ammonium Acetate, 10 mM Magnesium Acetate, 1mM EDTA, 0.1% SDS, pH 8.0). The gel-suspension was vortexed and ssDNA solution removed, leaving gel aside. An equal volume of phenol/chloroform/isoamyl alcohol (25:24:1) was mixed with ssDNA solution and centrifuged for 2 minutes at 16,000×g to recover supernatant. 2.5 volumes of ice-cold Ethanol (>99.8%) and 0.1 volume of 3 M NaCl was added with ssDNA solution to permit complete precipitation, incubated at −20° C. for 2 hours. The mixture was then centrifuged at 16,000×g for 30 minutes at 4° C. and the pellet washed with 90% ethanol and centrifuged at 16,000×g for 10 minutes to recover the ssDNA pellet. The pellet was held at 37° C. until dryness and later suspended in 10mM Tris HCl, pH 7.5 and stored at −20° C.

Enrichment assay of DNA pools: The enrichment of Neu5Gc binding DNA aptamers over the course of selection was monitored using a fluorescent dye linked aptamer assay. DNA pools obtained after each round, 1 to 10, were examined. Briefly, 100 pmol of sp-biotin or Neu5Gc-sp-biotin were immobilized to 250 μg of MyOne streptavidin C1 Dynabeads in PBS, pH 7.4 at room temperature for 30 minutes, followed by washing in PBS. ssDNA pool (˜60 ng) from each SELEX round in binding buffer was denatured and renatured as described above and incubated with target coated Dynabeads for 1 hour at 37° C., after which the Dynabeads were washed twice in binding buffer. Target bound ssDNA was eluted in 50 μL of nuclease free water by heating beads at 95° C. for 5 minutes. Eluted ssDNA was incubated with 150 μL of OliGreen® prepared in nuclease free 10 mM Tris-HCl buffer (pH 7.5) in flat-bottom black opaque microtiter plates (Greiner BioOne, Belgium) and fluorescence determined at excitation 485 nm and emission 520 nm, using a SpectraMax M5 reader.

Cloning and sequencing of enriched DNA pools: DNA pools were selected for cloning into pCR4.0 vector (TOPO TA cloning kit, Invitrogen) and transformed into one-shot TOP10 (Invitrogen, USA). Clones were screened using blue-white screening and positive transformants were analysed using colony PCR, with randomly selected clones presented for sequenced (Eurofins MWG). For comparing DNA sequence diversity and conservation within the DNA pools, multiple sequence alignment was carried out using ClustalW 2 algorithm.

Specificity analysis of aptamer: Target Neu5Gc-sp-biotin, Neu5Ac-sp-biotin and sp-biotin (2 ug/mL) were incubated with free DNA aptamer (500 nM) for 1 hour at 37° C. on gentle rotation. Prior to incubation, each selected aptamer was prepared in binding buffer (PBS supplemented with addition of 5 mM MgCl₂ and 25 mM NaCl). The ssDNA was denatured at 95° C. for 5 minutes and subsequently cooled on ice for 10 minutes and before use permitted to reach room temperature. The ssDNA—biotinylated sugar complex was then incubated with streptavidin Dynabeads for 30 minute at room temperature with gentle rotation. The Dynabeads-complex was then washed 3 times with binding buffer. The bound ssDNA was then disrupted to permit elution into 50 uL of nuclease free water by heating at 95° C. for 5 minutes and fluorescence reading were measured of ssDNA with OliGreen® in TE buffer at excitation 485 nm and emission 520 nm to evaluate binding affinity.

Aptamer binding inhibition assay: Target Neu5Gc-sp-biotin (1 nmol) was bound with aptamers in presence of free Neu5Gc ranging from 0 to 1 mg/mL for 1 hour at 37° C. on gentle rotation. The concentration of aptamer was held constant at 750 nM. Prior to incubation, aptamer was prepared in binding buffer (PBS supplemented with addition of 5 mM MgCl₂ and 25 mM NaCl) for binding analysis by linearization, by heat denaturation at 95° C. for 5 minutes followed by immediate cooling on ice for 10 minutes and allowed to warm up at room temperature for 10 minutes. The ssDNA-sugar complex was then incubated with streptavidin Dynabeads for 30 minute at room temperature on gentle rotation. These Dynabeads complex were washed twice with binding buffer and eluted by disruption at 95° C. for 5 minutes in 50 uL of nuclease free water. Fluorescence measurement were taken to permit the evaluation of binding affinity with OliGreen® in TE buffer at excitation 485 nm and emission 520 nm, using a SpectraMax M5 reader.

Statistical analysis and Kd calculations: Statistical analysis was performed using Graphpad Prism version 5. All data represented as the average values of three replicates. The results were expressed as mean values with a standard error of mean (SEM). The Ka was calculated by fitting the data points by the non-linear regression analysis with the help of the following equation using GraphPad Prism software: Y=Bₘₐₓ×X/(K_{d}+X) where Y is specific binding, Bₘₐₓ is the maximum number of binding sites and X is concentration of aptamer.

Results/Outcomes

I. Generation of Novel scFv Molecules for Utilization in:

(i) The detection of Neu5Gc sialic acid form variant both bound and free in solution.

(ii) The discrimination of sialic acid form variants Neu5Gc from Neu5Ac.

(iii) The measurement and direct detection of total sialic acid, the combined sum of Neu5Gc and Neu5Ac.

(iv) The calculation of Neu5Ac content indirectly through calculation of points (iii) and (ii) above.

Immune response and library generation: Post immunization chicken serum antibody titration demonstrated a strong immune response against Neu5Gc, FIG. 1. Library generation of scFv fragments, comprising of either a short linker (SL) or a long linker (LL) between light and heavy change were prepared from the isolated spleen and marrow RNA, and presented into a phagemid vector for display as phage-scFv particles. Restriction digestion with BstNI demonstrated a high level of diversity within the generated scFv fragment library (FIG. 2) estimated diversity both short and long linker libraries is at ˜1.5×10⁷ variants.

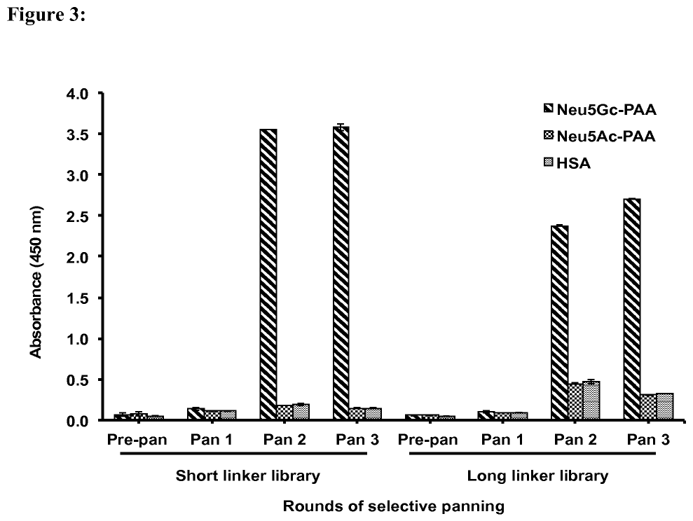

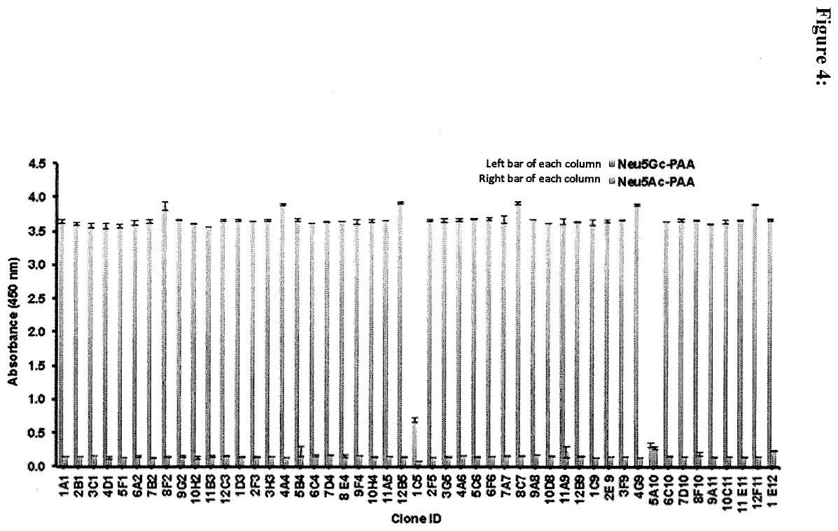

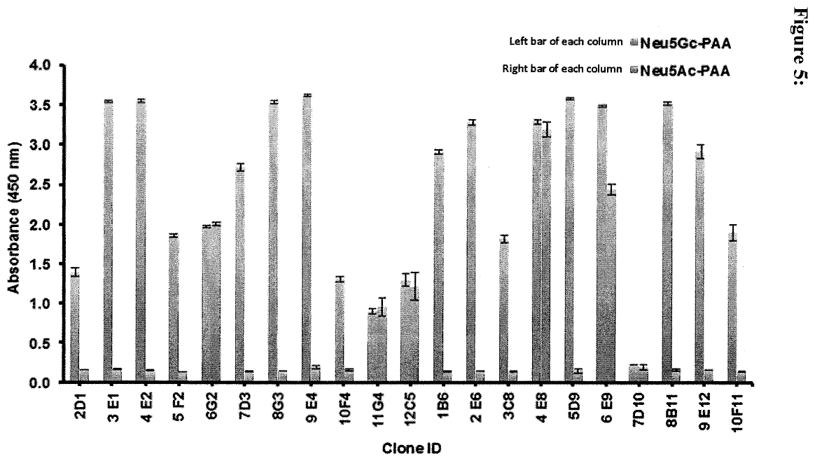

Identification and Isolation of anti-Neu5Gc specific phage and Neu5Ac cross reactivity: Both short and long linker libraries under went three rounds of biopanning against Neu5Gc-PAA. Amplified scFv-phage pools obtained post each round of biopanning were examined for recognition and specificity against Neu5Gc-PAA, Neu5Ac-PAA and HSA, and found to demonstrate a profile of high recognition and interaction with the Neu5Gc-PAA conjugate after rounds 2 and 3, for both SL and LL variants, FIG. 3. Minimal cross reactivity against Neu5Gc-PAA and HSA was observed across the ELISA. On the basis of ELISA, round 3 of biopanning was selected to proceed for the isolation and profiling of individual scFv-phage particles. 70 individual clones (49 SL clones and 21 LL clones) were randomly selected, cultured and analysed for anti-Neu5Gc specificity and Neu5Ac cross reactivity by ELISA, FIG. 4 and FIG. 5 respectively. Of the SL clones, virtually all the clones (97.9%) demonstrated high preferential binding specificity to Neu5Gc-PAA. Examination of the LL scFv-phage clone also demonstrated a high level of discrimination and specificity in 71.4% of the clones examined. Of note, a number of clones were found to demonstrate equal recognition and binding to both Neu5Gc and Neu5Ac in this presentation, clone NUIG_5A10 in FIG. 4, and clones NUIG_LL6G2, NUIG_LL11G4, NUIG_LL12C5, NUIG_LL4E8, NUIG_LL6E9 and NUIG_LL7D10) in FIG. 5. Demonstrating the generation of scFv which harbor the potential to be used for (i and ii) discrimination of Neu5Gc and also for the combined detection and analysis of Neu5Gc and Neu5Ac (iii), and therefore permitting measurement of Neu5Ac indirectly (iv) by subtraction of Neu5Gc content from total measured sialic acid content.

Sequence Analysis, homology assessment and consensus mapping: The scFv-phage individual clones underwent direct nucleotide sequencing to determine levels of diversity and homology, for the identification of putative key consensus regions active in the recognition and binding interaction to sialic acid variants. Post sequencing based on homology, high frequency and highly conserved sequences were observed for 2 unique SL variants (NUIG_SL1A1 and NUIG_SL4D1) and 3 SL variants (NUIG_LL1B6, NUIG_LL4E2 and NUIG_LLY) for clones specific to Neu5Gc. A consensus sequence demonstrating broad sialic acid recognition and affinity towards both Neu5Ac and Neu5Gc was also observed and reported here, NUIG_LLZ. scFv structures require the fusion of a light and heavy chain, typically performed using linker peptide, typically a glycine-serine (Gly₄Ser)₃ peptide. Within this work, linkers of 7 up to 21 amino acids where used and found to permit specific binding and discrimination with the following light and heavy chain combinations of Neu5Gc and total sialic acid (Neu5Ac and Neu5Gc).

Complete sequence of identified anti-Neu5Gc specific scFv light chains:

A. NUIG_SLIA1 light chain
MKKTAIAIAVALAGFATVAQAALTQPFSVSANLGETVKITCSGGSYAGSY
AYGWYQQKSPGSALVTVIYSNNKRPSDIPSRFSGSKSGSTGTLTITGVQA
EDEAVYYCGNEDSSDYFGAGTTLTVL
B. NUIG_SL4D1 light chain
MKKTAIAIAVALAGFATVAQAALTQPSSVSANPGGTVKITCSGSSSRSYG
WYQQKSPGSAPVTLIYENDKRPSDIPSRFSGSKSGSTGTLTITGVQAEDE
AVYYCGNEDSSDYFGAGTTLTVL
C. NUIG_LL1B6 light chain
MKKTAIAIAVALAGFATVAQAALTQPSSVSANPGETVKITCSGGHSSYYG
WYQQKSPGSAPVTVIYSNNQRPSDIPSRFSGCKSGSTNTLTITGVRADDE
AVYYCGGEDSSGAGHVAIFGAGTTLTVL
D. NUIG_LL4E2 light chain
MKKTAIAIAVALAGFATVAQAALTQPSSVSANLGGTVKITCSGGSSYYGW
YQQRSPGSAPVTVIYANNQRPSDIPSRFSGSKSGSTDTLTITGVRAEDEA
VYSCGSEDSSGAGHVAIFGAGTTLTVL
E. NUIG_LLY light chain
MKKTAIAIAVALAGFATVAQAALTQPSSVSANPGGTVKITCSGSSGSYGW
YQQKSPGSAPVTVIYYNDKRPSDIPSRFSGSKSGSTGTLTITGVQAEDEA
VYYCGNEDSSDYFGAGTTLTVL

Complete sequence of identified anti-sialic acid (Neu5Ac and Neu5Gc) scFv light chains:

F. NUIG_LLZ light chain
MKKTAIAIAVALAGFATVAQAALTQPSSVSANPGETVKITCSGGDYGSYY
GWYQQKSPGNTLVTLIYYNDKRPSNIPSRFSGSKSGSTGTLTITGVQAED
EAVYYCGSRDSSYVGIFGAGTTLTVL

Complete sequence of identified anti-Neu5Gc specific scFv heavy chains:

G. NUIG_SLIA1 heavy chain
AVTLDESGGGLQTPGGTISLVCKASGFDFSSHGMMWVRQAPGKGLEYVAA
INKHSEATWYGAAVKGRATISRDNGQSTVRLQLNNLRAEDTATYYCAKTD
YPGFNVGRIDAWGHGTEVIVSSTSGQAGQHHHHHHGAYPYDVPDYAS
H. NUIG_SL4D1 heavy chain
AVTLDESGGGLQTPGGALSLVCKASGFTFSDYGMMWVRQAPGKGLEYVAA
INRHSEATWHGAAVKGRATISRDNGQSTVRLQLNNLRAEDTATYYCAKTD
YSGFNVGRIDAWGHGTEVIVSSTSGQAGQHHHHHHGAYPYDVPDYAS
I. NUIG_LL1B6 heavy chain
AVTLDESGGGLQTPGGGLSLVCKASGFTFSSHGMFWVRQTPGKGLEYVAA
INSIGSYTYYGPAVKGRATISRDNGQSTVRLQLNNLRAEDTGTYYCAKDS
TSGYHIGRIDAWGHGTEVIVSSTSGQAGQHHHHHHGAYPYDVPDYAS
J. NUIG_LL4E2 heavy chain
AVTLDESGGGLQTPGGGLSLVCKASGFTFSSHGMLWVRQTPGKGLEYVAA
INSVGSYTYYGPAVKGRATISRDNGQSTVRLQLNNLRAEDTATYYCAKDS
SSGYHLGRIDAWGHGTEVIVSSTSGQAGQHHHHHHGAYPYDVPDYAS
K. NUIG_LLY heavy chain
AVTLDESGGGLQTPGGGLSLVCKASGFTFSDYGMMWVRQAPGKGLEYVAA
INRDSGATWYGAAVKGRATISRDNGQSTVRLQLNNLRAEDTATYYCAKTD
YSGFNVARIDPWGHGTEVIVSSTSGQAGQHHHHHHGAYPYDVPDYAS

Complete sequence of identified anti-sialic acid (Neu5Ac and Neu5Gc) scFv heavy chains:

L. NUIG_LLZ heavy chain
AVTLDEAFFGTAEDIAARALQPSGGLEFIRSFRKASEDQRVNLPPNLAQL
VHSAAYGRSEDSRVWRYFYDLAASPLHPFRGGSFRWAPYRPSRTEVIVSS
TSGQAGQHHHHHHGAYPYDVPDYAS

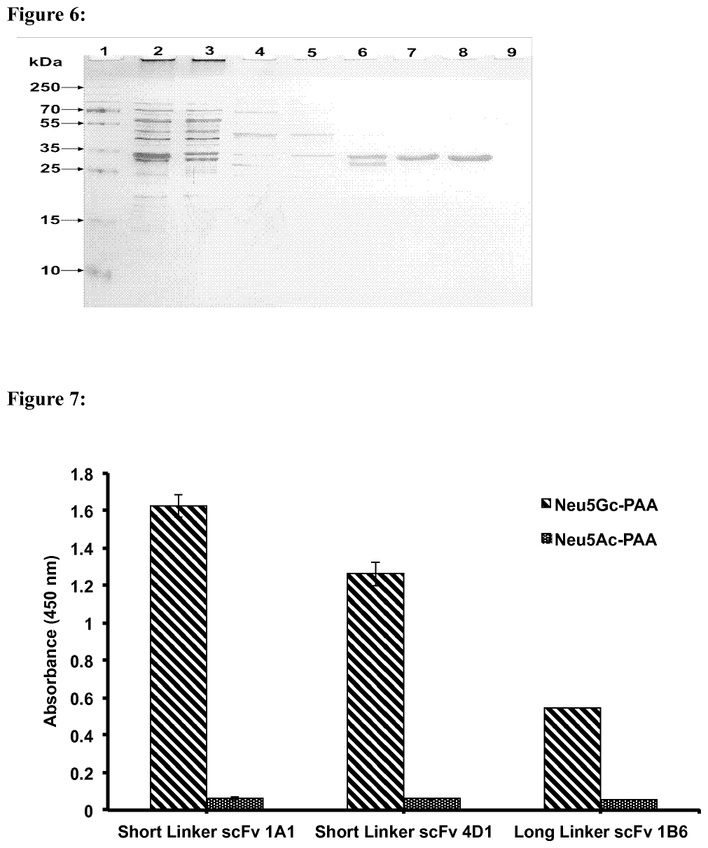

Expression and Purification of Soluble scFv: Phagemid from the clones representative of highly conserved sequences from both the SL and LL variants (NUIG_SL1A1 and NUIG_SL4D1; NUIG_LL1B6, NUIG_LL4E2 and NUIG_LLY) were expressed in E. coli TOP10 F′ cells under Isopropyl β-D-1-thiogalactopyranoside (IPTG) induction, for soluble scFv production. After soluble expression (overnight at 30° C. with rotation), individual soluble scFv was isolated and affinity purification (Ni-NTA affinity chromatography) for downstream applications. Purity of protein preparations and purifications was performed by reducing SDS-page, with visualization by silver stain (FIG. 6) and quantified by Bradford assay. Analysis of soluble scFv by direct ELISA: Analysed in a direct ELISA format for anti-Neu5Gc recognition soluble expressed and purified scFv, specifically detected Neu5Gc, with minimal binding close to background was observed shown for NUIG_SL1A1, NUIG_SL4D1 and NUIG_LL1B6, with Neu5Ac (as negative control), FIG. 7.

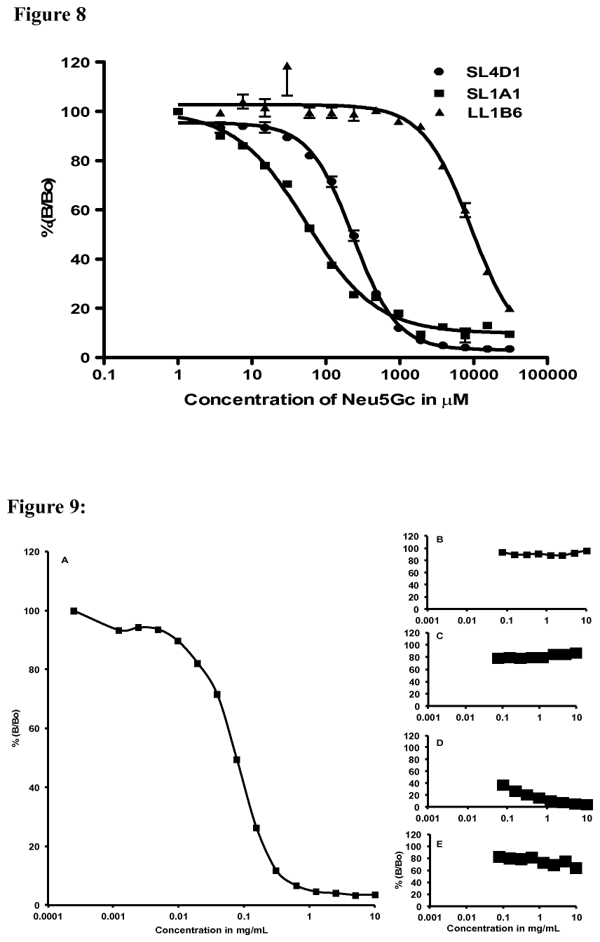

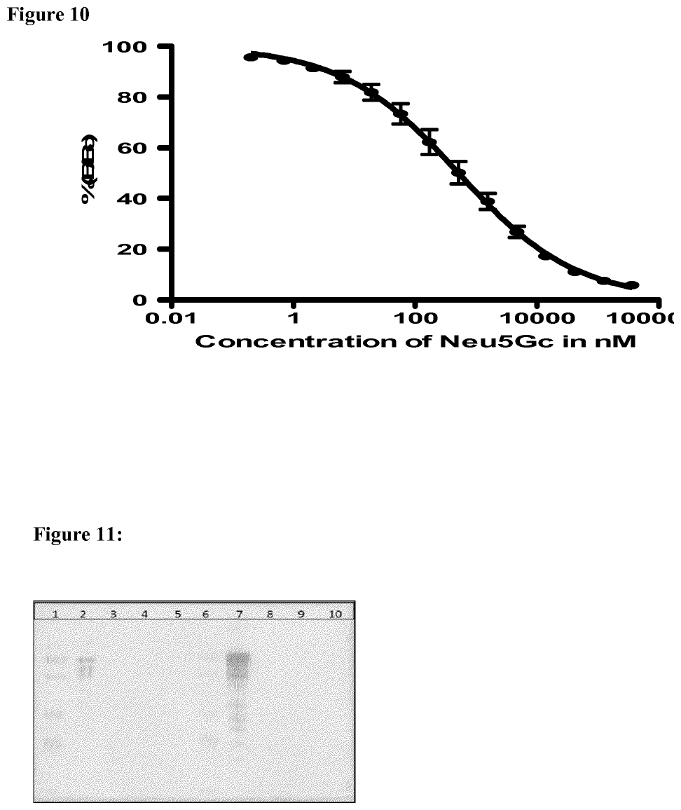

Competitive ELISA and specificity testing of scFv: scFv variants were tested in a competitive ELISA format, with NUIG_SL1A1 scFv demonstrating a high degree of sensitivity for free Neu5Gc inhibition, FIG. 8. NUIG_SL1A1 scFv was further evaluated for specificity, tested against glycoconjugates and glycoproteins with or without Neu5Gc present. Detection and quantification of Neu5Gc on the glycoprotein fetuin as compared to an asialofetuin control (fetuin presented with sialic acid chemically removed). Similarly, no response for Neu5Ac was observed, FIG. 9. Demonstrating NUIG_SL1A1 is capable to detect Neu5Gc both as a glycoconjugate and in its natural presentation on glycoproteins. A composite standard cure, FIG. 10, from independent assays produced an IC₅₀ of 221.3 μM for scFv SL1A1.

Neu5Gc detection on immunoblot and analytical validation: NUIG_SL1A1 was further assessed to detect Neu5Gc on protein immunoblots. Proteins, fetuin and transferrin of human and bovine source, both in naïve state and post acid treatment, were blotted onto PVDF membrane after reducing SDS-page under standard conditions. NUIG_SL1A1 demonstrated binding and detection of both bovine fetuin and bovine transferrin, indicating that both of these molecules in naïve form carry Neu5Gc. Human transferrin and asialofetuin did not result in any response consistent with fact that these proteins should not contain Neu5Gc. Confirmation was performed by re-assessment of samples post acid treatment (1M HCL for 2 hours at 80° C.) for removal of sialic acid. Both acid treated and non-treated proteins were examined by immunoblot (PVDF) in parallel. NUIG_SL1A1 demonstrated interaction with only non-acid treated fetuin and bovine transferrin as before supporting that signal was specific to sialic acid presence, FIG. 11. scFv specific to Neu5Gc had there affinity profiles assessed by SPR, returning affinity values (KD) in the range of 10⁻⁸ to 10⁻¹⁰ subject to specific clones and combinations of the claimed sequences contained.

II. Generation of Novel DNA Aptamer's for Utilization in:
(i) The specific discrimination ofNeu5Gc and Neu5Ac sialic acid forms
(ii) The measurement and direct detection ofNeu5Gc sialic acid
(iii) The measurement and direct detection of Neu5Ac sialic acid
(iv) The measurement and determination of total sialic acid, directly through calculation of points (ii) and (iii) in combination

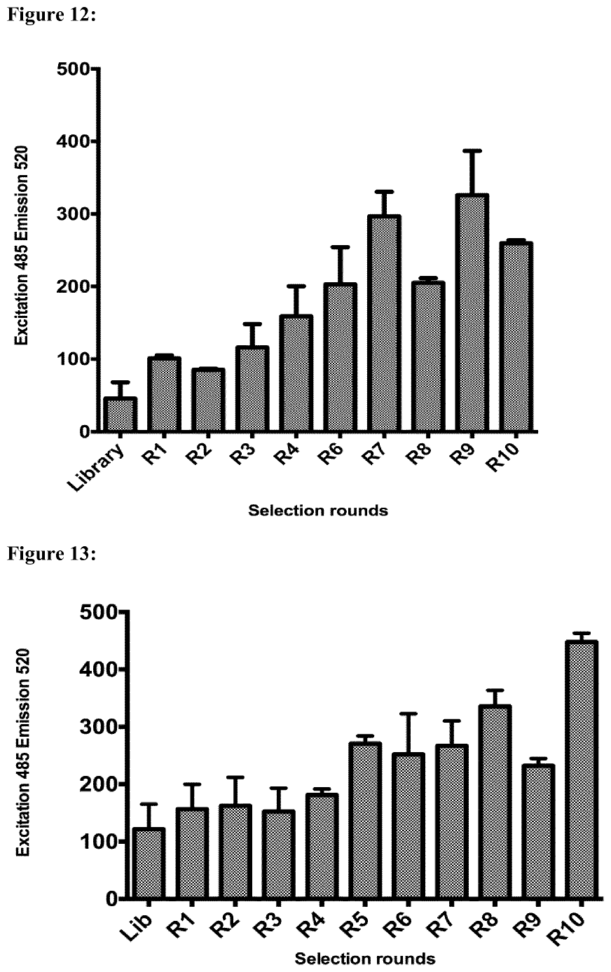

Aptamer Selection and sequence analysis: Enrichment of recognition binding to the target sialic acid conjugate was assessed over the ten positive SELEX rounds for both (i) Neu5Gc-sp-biotin, FIG. 12 and (ii) Neu5Ac-sp-biotin, FIG. 13, a fluorescent assay using OliGreen® stain to quantitate ssDNA us. Indicated by OliGreen®, the rounds of aptamer selection 7 and 9 where selected for (i) Neu5Gc-sp-biotin and rounds 8 and 10 Neu5Ac-sp-biotin TOPO cloning and direct sequencing.

A total of 482 aptamer single clones (218 isolated from round 7, 250 isolated from round 9) against Neu5Gc-sp-biotin were analysed, of which 468 (97.1%) returned were found to be within the acceptance of full-length (-60 bp). Three highly conserved sequences NUIG_GC-101, NUIG_GC-111 and NUIG_GC-224 were identified following detailed analysis, which covered 97.1% of the returned sequence data (Table 5).

These three sequences also permit the generation of a highly conserved single sequence which corresponds to the same frequency of occurrence (Table 5). A total of 189 aptamer single clones representing isolates from round 8 and round 10 against Neu5Ac-sp-biotin were analysed, of which 164 (86.7%) returned were found to be within the acceptance of full-length (˜60 bp). Three highly conserved sequences NUIG_AC-79 (22.2%), NUIG_AC-89-94 (35.9%) and NUIG_AC-93-68 (28.6%) were identified following detailed analysis, which when combined covered 86.7% of the returned sequence data as selected against Neu5Ac-sp-biotin (Table 6). These three sequences also permit the generation of a highly conserved single sequence which corresponds to the same frequency of occurrence (Table 6).

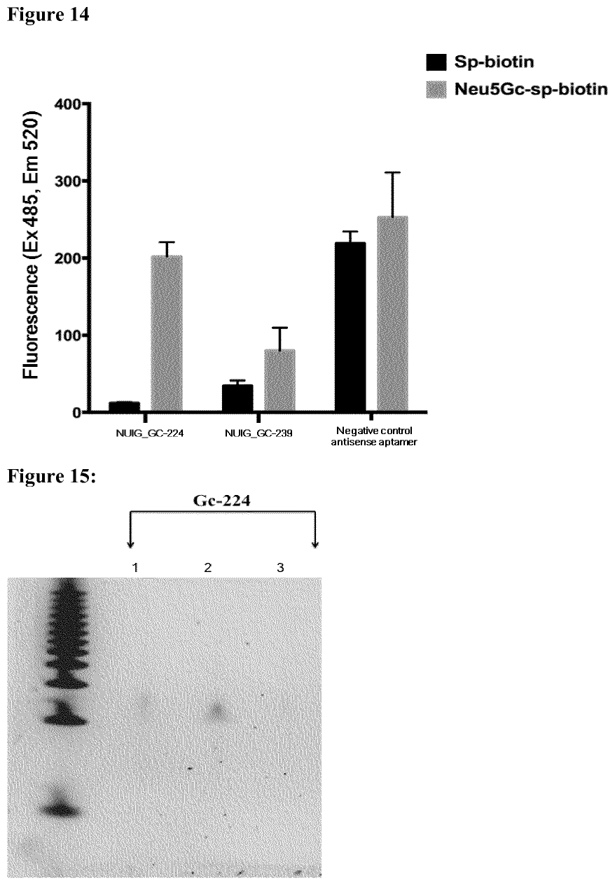

Evaluation of binding of consensus aptamer sequence: Neu5Gc-sp-biotin aptamer sequences were individual synthesis as 60-mer oligonucleotides and assessed for binding on ELISA based format against Neu5Gc-sp-biotin as bound antigen (FIG. 14). Representing the conserved aptamer sequences, NUIG_GC-224 and at a low frequency NUIG_GC-239 (Round 9; 0.016%) selective binding is demonstrated when compared against a selected negative control antisense aptamer, at matched concentrations. Levels of interaction with non-specific binding (sp-biotin) and against the bound target (Neu5Gc-sp-biotin) can be seen with the negative control antisense aptamer, FIG. 14, which are reduced to non-specific background assay levels for the NUIG_GC-224 and NUIG_GC-239 aptamers.

Aptamer NUIG_GC-224 was tested on urea gels to examine and demonstrate binding specificity to Neu5Gc-spacer-biotin, and to demonstrate specificity and ability to discriminate sialic acid forms, tested against Neu5Ac-spacer-biotin. For background, binding against sp-biotin was performed to assess potential binding and also form presented differences. Briefly, the biotinylated structures were immobilised on to the streptavidin beads and incubated with aptamer NUIG_GC-224. After washing, bound aptamers were eluted and loaded on urea gels for visualization, stained with SYBR Safe (Thermofisher, UK). The aptamer NUIG_GC-224 demonstrated a band within the captured Neu5Gc-spacer-biotin lane. No bands were observed for sp-biotin capture, and only a minimal signal observed in the Neu5Ac-spacer-biotin lane, (FIG. 15). This demonstrated and supports the claim of a specific DNA aptamer for Neu5Gc recognition and interaction.

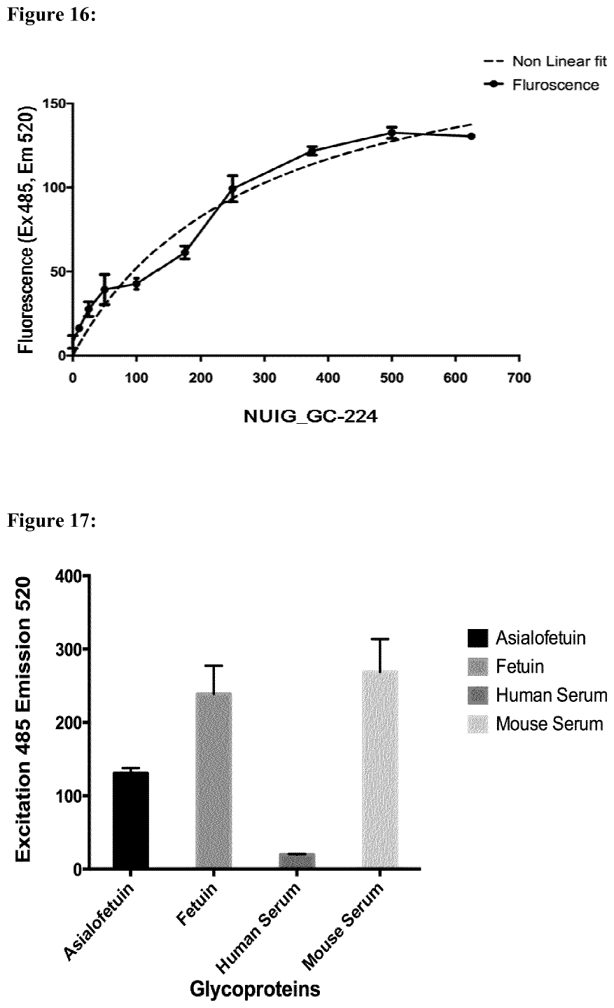

Determination of binding affinities aptamers against Neu5Gc and Neu5Ac: Binding curves were established at aptamer concentrations of 0 up to 1000 nM and Kd values estimated by linear regression. Binding intensity reached saturation at an aptamer concentration of ˜500 nM. Kd determination for NUIG_GC-224 was estimated as 2.83* 10⁷ M⁻¹ with a R² value of 0.95, FIG. 16.

Detection of Neu5Gc in glycoprotein sample: Determination of the ability and suitability of NUIG_GC-224 for the recognition and therefore the detection of Neu5Gc in its native state on glycoproteins, was assessed utilising a panel of glycoprotein. Both fetuin and asialo-fetuin were biotinylated to facilitate immobilization on the streptavidin-coated magnetic beads. Aliquots of mouse and human serum, complex glycoprotein pools, were similarly biotinylated, confirmed by direct ELISA.

Upon examination and testing with NUIG_GC-224 higher binding was observed with fetuin than asialofetuin, as would be anticipated, FIG. 17, due to the number of sialic acid moieties removed during the preparation of asialofetuin. NUIG_GC-224 showed significantly higher binding to the biotinylated mouse serum protein pool (Neu5Gc and Neu5Ac) than the human serum protein pool (Neu5Ac only), FIG. 17.

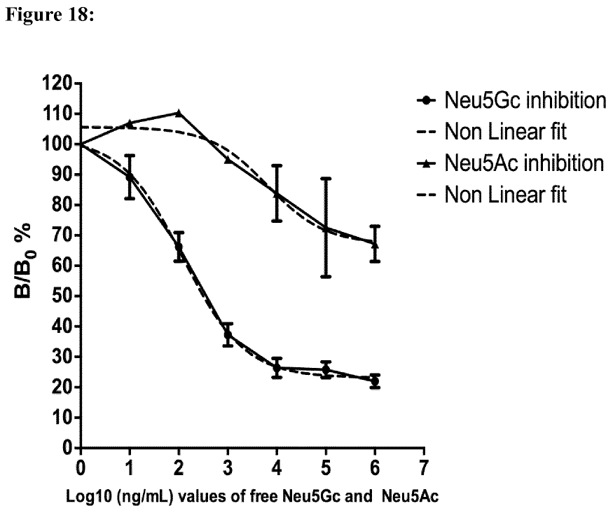

Development of an aptamer based competitive binding assay: A competitive binding assay was established with NUIG_GC-224 and free Neu5Gc as standard over an assay concentration range of 10 ng/mL to 1 mg/mL. This produced a standard curve that was linear over the Neu5Gc concentration range 10 ng/mL to 10 ug/mL and had an IC₅₀ value of 121 ng/mL (FIG. 18). These results indicate the potential specificity of the NUIG_GC-224 competitive assay for Neu5Gc for adaptation into a quantitative assay. In parallel, Neu5Ac was tested in the assay over the same concentration range, producing a 30% inhibition of binding (B/B₀=70%). This interaction is possible to overcome with tightening of the sensitivity range of assay development.

Evaluation of NUIG_GC-224 aptamer specificity: Neu5Gc-Sp-biotin (2 ug/m1) was immobilized onto magnetic dynabeads and later, NUIG_GC-224 along with equal concentrations [1 mg/mL] of free monosaccharides; Neu5Gc, Glucose, Galactose, Fucose, Mannose, Xylose and a PBS control, where added to observe potential interactions. The binding fluorescence intensity of Neu5Gc-Sp-biotin with NUIG_GC-224 without any inhibition was considered as 100% binding. Free Glucose, Galactose and Mannose showed did not significantly impact the binding with greater than 90% observed, Fucose and Xylose showed 71.7% and 75.7% binding respectively. Free Neu5Gc however, resulted in the diminishment of binding signal to 23.3%, showing that a concentration of lmg/mL inhibited 77.6% of NUIG_GC-224 binding with Neu5Gc-sp-biotin.

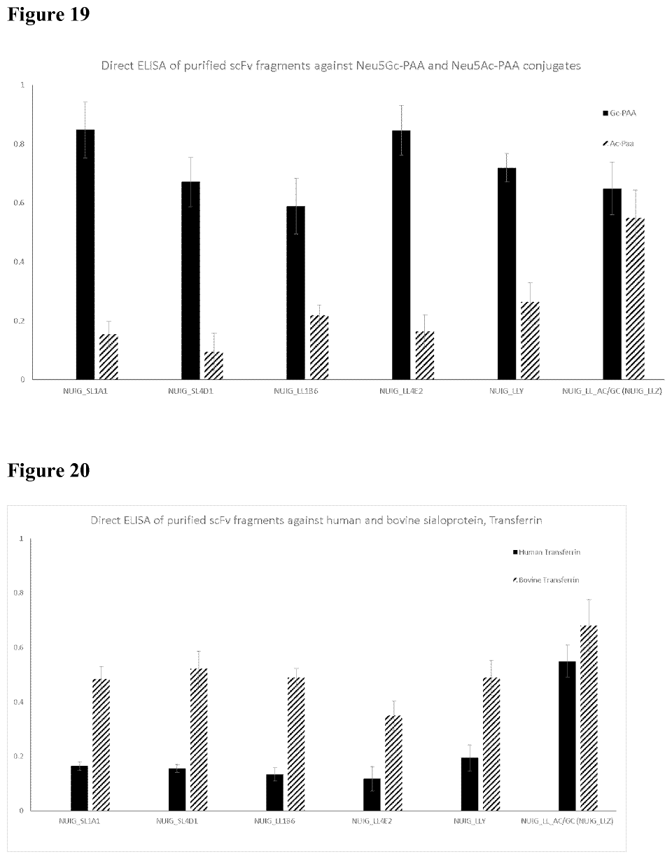

FIG. 19 demonstrates the results of an ELISA assay showing the discrimination of Neu5Gc from Neu5Ac for individual scFv preparations (NUIG_SL1A1, NUIG_SL4D1, NUIG_LL1B6, NUIG_LL4E2, and NUIG_LLY). Only one, NUIG_LL_AC/GC is found to recognise and interact with both sialic acid presentations. Neu5Gc and Neu5Ac are presented here as PAA conjugates which is indicative of detection of free form sialic acid.

FIG. 20 demonstrates the results of an ELISA assay showing the discrimination of Neu5Gc from Neu5Ac for individual scFv preparations (NUIG_SL1A1, NUIG_SL4D1, NUIG_LL1B6, NUIG_LL4E2, and NUIG_LLY). Only one, NUIG_LL_AC/GC is found to recognise and interact with both sialic acid presentations. Neu5Gc and Neu5Ac are presented here carried on a whole protein as they would commonly be presented within biological systems. In this instance, the protein transferrin from both human and bovine sources is used, where Neu5Ac sialic acid would only be expected to be observed on human, while bovine transferrin would carry both Neu5Ac and also Neu5Gc, which is indicative of detection of protein presented sialic acid.

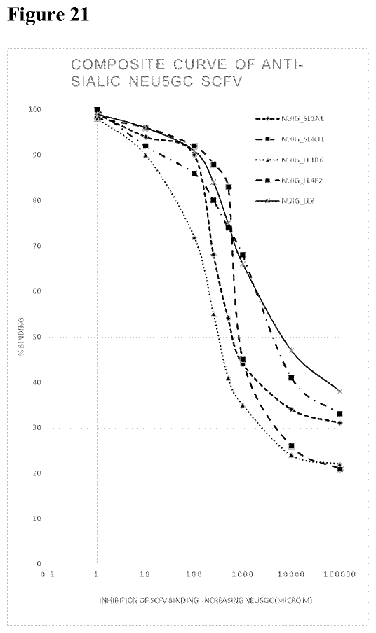

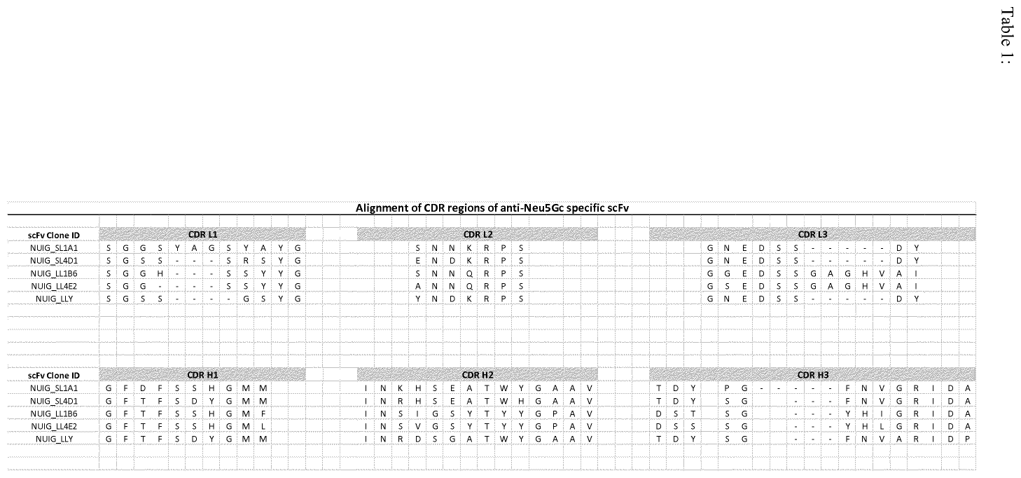

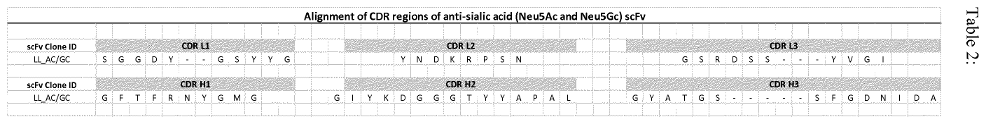

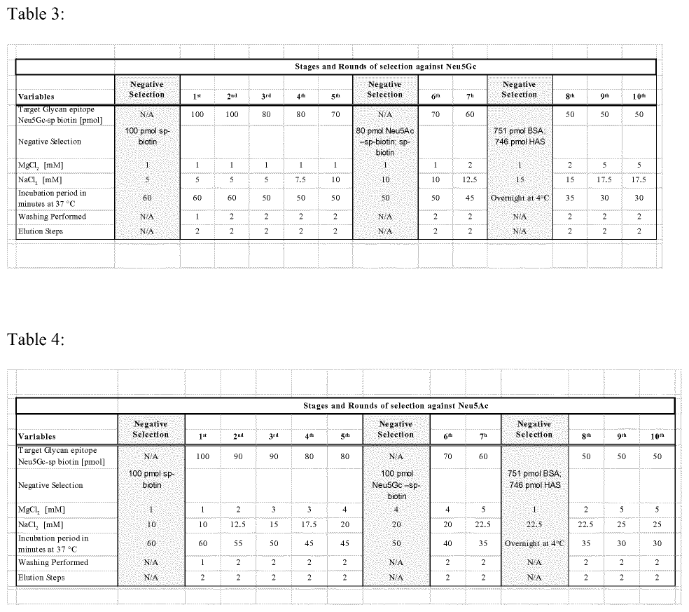

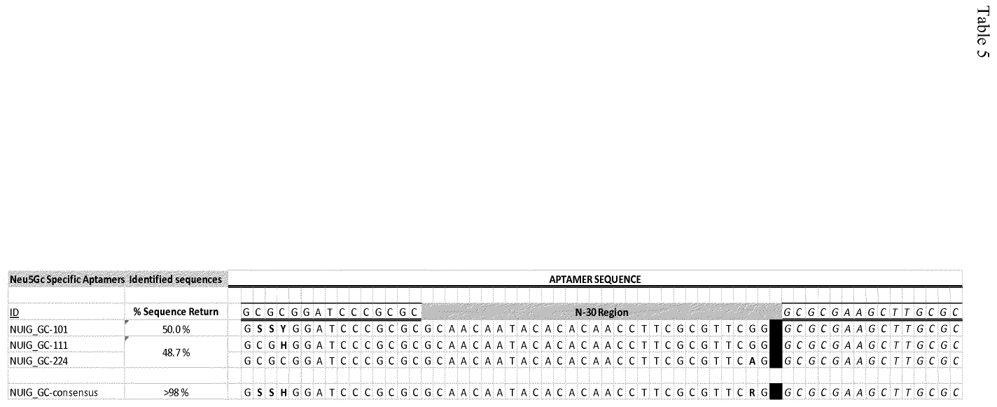

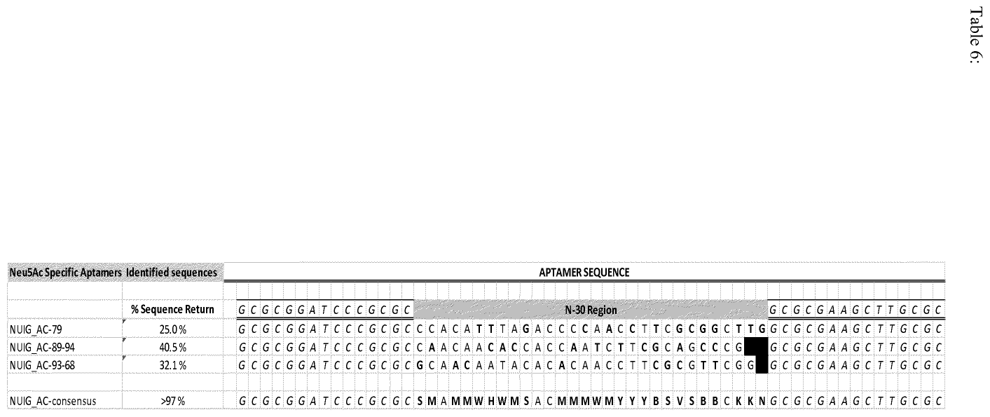

FIG. 21 demonstrates the results of a competitive ELISA generated composite curve for the Neu5Gc specific scFv molecules (NUIG_SL1A1, NUIG_SL4D1, NUIG_LL1B6, NUIG_LL4E2, and NUIG_LLY). An ELISA was performed in the presence of increasing free sialic acid (Neu5Gc) to compete/inhibit the binding of the scFv to the presented fixed concentration of bovine transferrin (source of Neu5Gc) permitting an inhibition curve to be generated permitting inhibitory concentrations to be determined and also presented for the determination of unknown samples from the curve, based on their inhibition level.

Findings:

Presented here are novel protein and nucleic acid molecules, of high affinity and high specificity towards the sialic acid Neu5Gc and Neu5Ac variants. Specificity and suitability of protein and nucleic acid molecules for utilisation in in vitro binding assays has been supported.

The invention provides anti-sialic acid recognition molecules for detection and the specific discrimination of non-human sialic acid Neu5Gc from the human Neu5Ac form and application in biological research, autoimmune disease and oncology fields, throughout the biopharmaceutical industry and as potential candidate pipeline molecules for future therapeutic intervention of disease and as delivery vehicles against cell and tissues expressing Neu5Gc.

In the present invention anti-Neu5Gc molecules and non-discriminatory molecules have been identified by (i) phage display (scFv) and (ii) SELEX (aptamer) technology. The anti-Neu5Gc scFv molecules generated from immune challenged chickens demonstrate specific detection and discrimination of Neu5Gc from Neu5Ac in free, as well as in bound form. Further to this, non-discriminatory scFv molecules of high affinity against total sialic acid have been identified (equally interacting with Neu5Ac and Neu5Gc). The development of aptamers with specificity against Neu5Gc and Neu5Ac allowing for the unique discrimination of Neu5Gc and Neu5Ac has also been achieved. Both recognition molecule types (scFv or aptamer) can be utilized over a wide range of ligand based assays for the detection, quantification of total sialic acid and the discrimination of Neu5Gc and Neu5Ac. The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

1. A sialic acid recognition molecule selected from (i) a recombinant single chain variable fragment (scFv) antibody or (ii) a DNA oligonucleotide aptamer.

2. A sialic acid recognition molecule as claimed in claim 1 which is an Anti-Neu5Gc recognition molecule, the molecule being a recombinant anti-Neu5Gc antibody which is a single chain variable fragment, comprising a variable light chain sequence specific for anti-Neu5Gc scFv comprising the following sequences:
Sequence (1): SG-X¹-X²-X³-X⁴-X⁵-X⁶-X⁷-X⁸-YG, wherein,
X¹ is G or S;
X² is S of H;
X³ is Y or absent;
X⁴ is A or absent;
X⁵ is G or absent;
X⁶ is S or absent;
X⁷ is S or Y or R or G;
X⁸ is Y or A or S,
and Sequence (2): X¹-NDKRPS, wherein,
X¹ is A or S or E or Y,
and either Sequence (3): G-X¹-EDSSGAGHVAI, wherein,
X¹ is S or G, or Sequence (4): GNEDSSDY.

3. A sialic acid recognition molecule as claimed in claim 1 which is an Anti-Neu5Gc recognition molecule, the molecule being a recombinant anti-Neu5Gc antibody comprising a variable heavy chain sequence specific for anti-Neu5Gc scFv comprising the following sequences:
Sequence (5): GF-X¹-FS-X²-X³-GM-X⁴, wherein,
X¹ is T or D;
X² is D or S;
X³ is Y or H;
X⁴ is M or L or F,
and Sequence (6): YVAAIN-X¹-X²-X³-X⁴-X⁵-T-X⁶-X⁷-G-V-AV, wherein,
X¹ is R or K or S;
X² is H or D or V or I;
X³ is S or G;
X⁴ is E or G or S;
X⁵ is A or Y;
X⁶ isWorY;
X⁷ is H or Y;
X⁸ is A or P,
and either Sequence (7): TYYCAKTDY-X¹- GFNVGRID- X²,
wherein, X¹ is S or P; X² is A or P,
or Sequence (8) TYYCAKDS-X¹-SGYH-X²-GRIDA,
wherein, X¹ is S or T; X² is L or I.

4. A total anti-sialic acid single chain variable fragment (scFv) antibody fragments, which recognises total (both Neu5Gc and Neu5Ac sialic acid forms), comprising a variable light chain sequence and a variable heavy chain sequence.

5. A total anti-sialic acid single chain variable fragment (scFv) as claimed in claim 4 wherein the variable light chain sequence comprises at least two of the sequences:
(9) SGGDYGSYYG,
(10) YNDKRPSN, or
(11) GSRDSSYVGI.

6. A total anti-sialic acid single chain variable fragment (scFv) as claimed in claim 4 wherein the variable heavy chain sequence may comprise at least two of the sequences:
(12) GFTFRNYGMG,
(13) GIYKDGGGTYYAPAL, or
(14) GYATGSSFGDNIDA.

7. A recombinant single chain variable fragment (scFv) antibody as claimed in claim 4 further comprising a polypeptide linker sequence having the formula:
(15) GQSSRSS-Xⁿ(G4S2)n)
wherein, X may be absent or GGGGSS, or GGGGSSGGGGSS.

8. A sialic acid recognition DNA oligonucleotide aptamer molecule(s) as claimed in claim 1 which are for either anti-Neu5Gc or anti-Neu5Ac selected from the following sequences:
anti-Neu5Gc DNA oligonucleotide aptamer molecule(s):
(16) 5′-CGCGCGCAACAATACACACAACCTTCGCGTTCRG-3′, where R is either G or A:
anti-Neu5Ac DNA oligonucleotide aptamer molecule(s):
(17): 5′-CGCGCCCACATTTAGACCCCAACCTTCGCGGCTTG-3′, or
(18): 5′-CGCGCCAACAACACCACCAATCTTCGCAGCCCG -3′, or
(19): 5′-CGCGCGCAACAATACACACAACCTTCGCGTTCGG -3′, or comprising a consensus sequence:
(20): 5′-SMAMMWHWMSACMMMWMYYYBSVSBBCK -3′,
where S is either C or G; M is either C or A; W is either T or A; H is either T, C or A; Y is either C or T; B is either T, G or C; V is either G, A or C; K is either T or G.

9. A nucleic acid or oligonucleotide sequence as claimed in claim 1 which are substantially similar to the said sequences and which also bind to Neu5Gc and/or Neu5Ac, and which have at least 85% sequence identity with these sequences, or which are complimentary to those sequences.

10. The sialic acid recognition molecule of claim 1 and comprising a detectable label.

11. The sialic acid recognition molecule of claim 1 and a cytotoxic moiety.

12. A method of determination and quantification of Neu5Gc comprising use of the sialic acid recognition molecule of claim 1.

13. A method of determination and quantification of total sialic acid and discrimination of Neu5Ac (inferred) and Neu5Gc comprising use of the sialic acid recognition molecule of claim 1.

14. An assay for the determination of the presence or the quantification of a total sialic acid, and discrimination of Neu5Ac or Neu5Gc motif in tissues or cells or on proteins, comprising the use of the sialic acid recognition molecule of claim 1.

15. A method of determining the inferred quantification of Neu5Ac and or Neu5Gc comprising use of the sialic acid recognition molecule of claim 1.

16. A peptide sequence selected from sequences A to L as defined herein.
